(19) United States
(12) Patent Application Publication
(10) Pub. No.: US 2016/0158408 A1
(43) Pub. Date: Jun. 9, 2016

(54) IRRIGATION RESISTANT COMPOSITIONS FOR REGENERATION OF HARD TISSUES AND METHODS OF USING THE SAME

(71) Applicant: NOVABONE PRODUCTS, LLC, Alachua, FL (US)

(72) Inventors: Gregory J. Pomrink, Newberry, FL (US); Zehra Tosun, Gainesville, FL (US)

(21) Appl. No.: 15/044,799

(22) Filed: Feb. 16, 2016

Related U.S. Application Data
(63) Continuation-in-part of application No. 14/512,976, filed on Oct. 13, 2014.
Application No. 14/512,976 is a continuation-in-part of application No. 14/369,119, filed on Jun. 26, 2014.
(60) Provisional application No. 61/738,585, filed on Dec. 18, 2012.
(60) Provisional application No. 61/787,827, filed on Mar. 15, 2013.

(51) Int. Cl. A61L 27/10; A61L 27/18; A61L 27/34; A61L 27/54; A61L 27/12

(52) U.S. Cl. CPC A61L 27/10; A61L 27/54; A61L 27/12; A61L 27/34; A61L 27/18; A61L 2430/02; A61L 2420/00; A61L 2400/06; A61L 2300/60; A61L 2300/112; A61L 2300/10

(57) ABSTRACT

An irrigation resistant bone repair composition including a biocompatible or bioactive bone repair material comprising borate, and poly(glycerol sebacate), wherein the composition, upon implantation into a surgical site, maintains position and does not displace upon irrigation of the surgical site, is described. Also, methods for treating a bone having a bone gap or a bone defect with the composition as well as kits that include the composition, are provided.

### RELATED APPLICATIONS

The present patent document is a continuation-in-part application of U.S. patent application Ser. No. 14/512,976, filed Oct. 13, 2014, which is a continuation-in-part application U.S. patent application Ser. No. 14/369,119, filed Jun. 26, 2014, which is §371 nationalization of International Application No. PCT/US2013/075741, filed Dec. 17, 2013, which claims the benefit of the filing date under 35 U.S.C. §119(e) of Provisional U.S. Patent Application Ser. Nos. 61/738,585, filed Dec. 18, 2012 and 61/787,827, filed Mar. 15, 2013, which are incorporated herein by reference in their entirety.

### BACKGROUND

Bone is a composite of collagen, cells, calcium hydroxyapatite crystals, and small quantities of other proteins of organic molecules that has unique properties of high strength, rigidity, and ability to adapt to varying loads. When bone injuries occur, it is necessary to fill voids or gaps in the bone as well as to encourage the repair and regeneration of bone tissue. There are many materials used today for the repair and regeneration of bone defects. For example, one material useful to encourage such repair and regeneration is bioactive glass.

Bioactive glass was originally developed in 1969 by L. Hench. Additionally, bioactive glasses were developed as bone replacement materials, with studies showing that bioactive glass can induce or aid in osteogenesis (Hench et al., J. Biomed. Mater. Res. 5:117-141 (1971)). Bioactive glass can form strong and stable bonds with bone (Piotrowski et al., J. Biomed. Mater. Res. 9:47-61(1975)). Further, bioactive glass is not considered toxic to bone or soft tissue from studies of in vitro and in vivo models (Wilson et al., J. Biomed. Mater. Res. 805-817 (1981)). Exemplary bioactive glasses include 45S5, 45S5B1, 58S, and 570C30. The original bioactive glass, 45S5, is melt-derived. Sol-gel derived glasses can also be produced and include nanopores that allow for increased surface area and bioactivity.

There are drawbacks to the use of bioactive glass or other materials in the form of liquids, pastes, and solids to fill voids or gaps in the bone. A liquid or a paste may not remain at the site of the void or gap in the bone. A solid may be difficult to apply and may not conform well to the void or gap in the bone. Solids may migrate or be displaced from the site through washing or other means.

These drawbacks may be reduced and/or eliminated by adding materials to a bone repair composition, such that the composition is rendered irrigation and migration resistant.

### SUMMARY

Certain embodiments relate to an irrigation resistant bone repair composition including a porous biocompatible or bioactive bone repair material comprising borate, and poly(glycerol sebacate), wherein the composition, upon implantation into a surgical site, maintains position and does not displace upon irrigation of the surgical site. The weight ratio of the poly(glycerol sebacate) may be 1%-99% relative to the weight of the bone repair composition; the weight ratio of the poly(glycerol sebacate) may be 1%-20% relative to the weight of the bone repair composition; the weight ratio of the poly(glycerol sebacate) may be 20%-30% relative to the weight of the bone repair composition; the weight ratio of the poly(glycerol sebacate) may be 30%-40% relative to the weight of the bone repair composition; the weight ratio of the poly(glycerol sebacate) may be 40%-50% relative to the weight of the bone repair composition; the weight ratio of the poly(glycerol sebacate) may be 50%-60% relative to the weight of the bone repair composition; the weight ratio of the poly(glycerol sebacate) may be 60%-70% relative to the weight of the bone repair composition; the weight ratio of the poly(glycerol sebacate) may be 70%-80% relative to the weight of the bone repair composition; or the weight ratio of the poly(glycerol sebacate) may be 80%-99% relative to the weight of the bone repair composition. The composition is osteoconductive. The composition is osteostimulative.

In the composition, the bone repair material is a borate bioactive glass or ceramic. The borate bioactive glass may be a melt-derived borate bioactive glass or a sol-gel derived borate bioactive glass. The bioactive glass may be crystalline, amorphous or a mixture and in the form of a particle, sphere, fiber, mesh, sheet or a combination of these forms. The composition may form a synthetic bone-grafting putty, paste, gel, strip or waxy solid. The composition may be in an injectable form. The bone repair composition promotes osteointegration upon introduction into a bony defect. In the bone repair compositions, the borate bioactive glass comprises about 0-45% CaO, about 10-70% B₂O₃, about 0-25% Na₂O, about 0-17% P₂O₅, about 0-30% MgO and about 0-5% CaF₂. Alternatively, in the bone repair composition, the borate bioactive glass comprises about 45% B₂O₃, about 24.5% CaO, about 6% P₂O₅, and about 2.5% Na₂O. The size of the bioactive glass particle may be in a range from about 0.01 μm to about 5 mm. In the compositions, the borate bioactive glass may include 0-80%<100 μm borate bioactive glass, 0-80%<500 μm borate bioactive glass, 0-80% 500-1000 μm borate bioactive glass, 0-80% 1000-2000 μm borate bioactive glass, 0-80% 2000-5000 μm borate bioactive glass, 0-90% 90-710 μm borate bioactive glass, and 0-90% 32-125 μm borate bioactive glass. In the bone repair composition, the bone repair material may be one or more particles of borate bioactive glass coated with a glycosaminoglycan, wherein the glycosaminoglycan is bound to the borate bioactive glass. The glycosaminoglycan may be selected from the group consisting of heparin, heparan sulfate, chondroitin sulfate, dermatan sulfate, keratan sulfate, and hyaluronic acid. The bone repair composition may further comprise at least one element selected from the group consisting of Li, K, Mg, Sr, Ti, Zr, Fe, Co, Cu, Zn, Al, Ag, Au, Ce, Ga, P, N, S, F, Cl, Si and I. The bioactive glass may be pretreated in a solution comprising one or more of blood, bone marrow, bone marrow concentrate, bone-morphogenetic proteins, platelet-rich plasma, and osteogenic proteins. The proteins may be selected from the group consisting of WP9QY(W9; YCWSQYLCY; SEQ ID NO:1), OP3-4, RANKL, B2A, P1, P2, P3, P4, P24, P15, TP508, OGP, PTH, NBD, CCGRP, (Asp)₆ (SEQ ID NO:2), (Asp)₈ (SEQ ID NO:3), and (Asp, Ser, Ser)₆ (SEQ ID NO:4), and mixtures thereof. The composition may include a bioactive agent, such as a bioactive glass ceramic comprising silica and boron. The composition may be in a flexible-crosslinked form in a shape of a strip. The bone repair composition may further comprise an additive selected from the group consisting of a solvent, a linear aliphatic hydrocarbon, straight chain aliphatic hydrocarbon, branched aliphatic hydrocarbon, sugar, polysaccharide, and hydroxyl terminal polyalkylene oxide, alkoxy terminal polyalkylene oxide, and a low molecular weight biodegradable polymers (MW</=10,000). The additive may be sodium hyaluronate, regenerez, polypropylene glycol 3000 (poly 3000), seasame oil, candelilla wax, carnauba wax, sorbitol (D-Glucitol), polycaprolactone, polycaprolactone diol, coconut oil, propylene glycol, polycaprolactone triol, polycaprolactone 10000 mw, mineral oil high viscosity, mineral oil low viscosity, polyethylene glycol 400, butylene glycol, and hexylene glycol.

Certain further embodiments relate to an irrigation resistant putty or paste including an irrigation resistant bone repair composition including a porous biocompatible or bioactive bone repair material comprising borate, and poly(glycerol sebacate) mixed with water, saline, blood, or BMA, wherein the irrigation resistant putty or paste, upon implantation into a surgical site, maintains position and does not displace upon irrigation of the surgical site.

Certain further embodiments relate to an irrigation resistant bone repair composition for treating a bone defect or a bone gap including a porous biocompatible or bioactive bone repair material comprising borate, and poly(glycerol sebacate).

Yet further embodiments relate to an irrigation resistant bone repair composition for regeneration of hard tissues including a porous biocompatible or bioactive bone repair material comprising borate, and poly(glycerol sebacate).

Certain further embodiments relate to a method for treating a bone having a bone gap or a bone defect comprising contacting the bone at or near the site of the bone defect with an irrigation resistant bone repair composition including a porous biocompatible or bioactive bone repair material comprising borate, and poly(glycerol sebacate).

### DETAILED DESCRIPTION

Irrigation resistant bone repair compositions comprising (i) a porous biocompatible or bioactive bone repair material comprising borate, and (ii) poly(glycerol sebacate), wherein the composition, upon implantation into a surgical site, maintains position and does not displace upon irrigation of the surgical site.

Specifically, certain embodiments relate to a synthetic bone grafting composition, such as a putty for bone repair that incorporates biodegradable elastomers, such as poly(glycerol sebacate) or other similar materials, having an osteoconductive, osteostimulative and irrigation resistant properties.

Among these elastomers, poly(glycerol sebacate) or PGS, which is well studied; it has the Young's modulus in the range of 0.05-1.5 MPa, with tuneable degradation kinetics as well (Chen Q-Z, et al., Biomaterials, 29(1): 47-57 (2008)) and ability to degrade quickly in vivo, being completely absorbed in less than 6 weeks. However, as discussed in S. L. Liang et al. (S.-L. Liang et al., Biomaterials, 31:8516-8529 (2010)), PGS has been previously found to produce cellular toxicity probably caused by non-reacted carboxylic acid groups or the carboxylic acids produced by aqueous hydrolysis of the PGS ester groups, which lower the local extracellular pH to below physiological values (7.2-7.4). To address these limitations, unique compositions of PGS and borate bioactive glass that are irrigation resistant, wherein upon implantation into a surgical site, the compositions maintain position and do not displace upon irrigation of the surgical site, are described.

The term “irrigation resistant” in connection with the compositions described herein refers to a property of the composition, where the composition can be heavily irrigated following placement in a surgical site without being washed away or displaced from the surgical site. The composition includes PGS, which is mixed with a biocompatible or bioactive bone repair material, such as bioactive glasses (e.g., bioactive glasses including borate) or other osteoconductive salts, glasses or ceramics for use in methods for treating a bone having a bone gap and/or a bone defect.

The irrigation resistant bone repair composition is biocompatible and or bioactive and comprised of entirely synthetic materials, eliminating any risk of disease transmission that may occur with products containing animal or human derived materials or components to achieve this property.

The composition promotes osseointegration when introduced into a bone gap and/or a bone defect.

The bone repair composition has a unique physical property of being irrigation resistant. The irrigation resistant characteristics provide a material, which maintains position in the surgical site despite the amount of blood, body fluid or saline to which it is exposed. Irrigation resistance is beneficial to simplify the application of the bone graft at the site of defect while preventing migration of the graft material during irrigation and after closure of the surgical site. The irrigation resistance of the bone repair composition is especially beneficial for its intended use in orthopedic and spine processes, as the material will stabilize and maintain placement and structure within the body during placement, irrigation and after closure. Specifically, in certain embodiments where a non-setting putty material is used, the bone repair composition will not be displaced easily during irrigation and closure of the surgical site.

An irrigation resistant, fully synthetic and bioactive putty, when implanted into the body, will maintain position or placement rather than melt, dissolve or disintegrate during irrigation or displace upon closure of the surgical site. This feature permits the implant to hold in place more easily, and create beneficial handling properties. The ability to resist displacement allows the bioactive agent to remain at the site of implantation to stimulate bone growth resistant to excessive irrigation of the surgical site prior to closure for an extended period of time (e.g., for up to at least 0.5 hours, up to at least 1 hour, up to at least 6 hours, up to at least 12 hours, up to at least 24 hours, up to at least 48 hours, up to at least 72 hours, but even for up to 7 days). The borate bioactive glass, as the preferred bioactive agent, stimulates the genes necessary to differentiate precursor cells into osteoblasts and the subsequent proliferation of these cells within the surgical site while undergoing an ionic exchange with the surrounding body fluid to form microcrystalline hydroxyapatite analogous to natural bone mineral. The combination of these properties in one composition is essential for bone regeneration and hard tissue repair.

The irrigation resistant composition may be a liquid at room temperature. Alternatively, the composition may have the consistency of a solid, gel, putty, paste or any other non-liquid substance at room temperature. The composition may also have the form of a liquid, solid, gel, putty, paste or any other non-liquid substance at temperatures other than room temperature. Additionally the composition may undergo a phase change when warmed from room temperature to body temperature.

The irrigation resistant bone repair composition provides for acceleration in the rate and an enhancement in the quality of newly-formed bone. Improved bone healing may occur in those who may be compromised, such as diabetics, smokers, the obese, the elderly, those who have osteoporosis, those who use steroids, and those who have infections or other diseases that reduce the rate of healing. The rapid hardening of the bone repair composition at the site of the bone defect can serve to localize the bone repair material, such as bioactive glass, at the site.

The bone repair composition may be provided to a site of a bone defect by means of a syringe or other injection device. In certain embodiments, the bone repair composition may be sufficiently liquid so as to be injectable, yet can harden suitably at the bone defect site at body temperature. For instance, if the bone repair composition is a liquid at room temperature, it may become a thick gel at body temperature; in other words, the bone repair composition can cure upon application to a bone defect at body temperature.

In certain embodiments, the bone repair composition has the advantages of low viscosity, runny liquid composition with regard to the ease of application to a bone defect site. Further advantages of the composition include more solid paste-like composition characteristics and that it remains positioned at the defect after being applied (i.e., is irrigation resistant). The solidification of the composition at body temperature overcomes the disadvantageous property of other liquid compositions that do not exhibit irrigation resistant behavior. At the same time, because the composition is not a solid at room temperature, there is greater ease of applying the composition, such as by means of a syringe. The composition need not be laboriously painted onto a bone defect or applied onto a bone defect by means of pressure.

Other delivery modes can be used for more viscous bone repair compositions. These modes include manually placing the gel or paste directly into a bone defect or extruding the gel or paste using a syringe, delivery gun or other means.

In certain embodiments, if the bone repair composition is a gel at room temperature, it may become a paste at body temperature.

In certain other embodiments, if the bone repair composition is a thick gel or paste at room temperature, it may become putty or a solid at body temperature.

The relative amount of PGS in the composition may determine the viscosity of the composition at room temperature and at body temperature. For example, 1-30% with the higher amounts yielding more viscous compositions.

In some embodiments, the weight ratio of PGS is 1%-99% relative to the weight of the bone repair composition, which is conversely 1-99% bioactive glass, such as borate bioactive glass.

This weight ratio may be from 1-10%, 10-20%, 20-30%, 30%-40%, 40%50%, 50%-60%, 60%-70%, 70%-80%, 80%-90%, or 90-99%. Alternatively, this weight ratio may be at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least at least about 97%, at least about 98%, or at least about 99%. The material may have the consistency of a solid, gel, putty, paste or any other non-liquid substance at room temperature.

The compositions may vary in molecular weight and be blended in ratios of 10:1 to 1:10.

The bone repair material can be any number of materials that assist in bone repair and production. Such materials include at least bioactive glass in the form of a particle, sphere, fiber, mesh, sheet or a combination of these forms, i.e. fibers within a sphere, and calcium salts, i.e., DCP, alpha TCP, beta-TCP, hydroxyapatite, calcium sulfates, calcium borates or calcium silicates, multiphasic calcium phosphates, calcium sulfates, calcium borates or calcium silicates along with elemental substitutions within these materials or coatings applied to these materials. In certain embodiments, the biocompatible or bioactive bone repair material may also be any combination of various therapeutic materials.

The various types of bioactive glass that may be used as bone repair material were previously described In U.S. Pub. No. US 2014/0079789, entire content of which is incorporated herein by reference. In certain preferred embodiments, borate bioactive glass is used. Bioactive glass may be melt-derived or sol-gel derived. Depending on their composition, bioactive glasses may bind to soft tissues, hard tissues, or both soft and hard tissues.

In certain preferred embodiments, the composition may be prepared as a composite with a biocompatible or bioactive agent, such as a bioactive glass ceramic, which contains boron. The ceramic releases calcium and boron ions, which facilitate the differentiation and proliferation of osteoblasts (defined as osteostimulation), which in turn increases the rate of regeneration of hard tissue. Furthermore, the composition of the bioactive glass may be adjusted to modulate the degree of bioactivity. For example, in certain embodiments, further advantage of borate is that is allows to control the rate of degradation.

In addition, the bioactive glass component undergoes an ion exchange with the surrounding body fluid to form hydroxyapatite analogous to bone mineral. More specifically, dissolution of the bioactive glass ceramics releases the calcium and boron ions, which stimulate the genes responsible of the differentiation and proliferation of osteoblast cells within the bony defect upon implantation. It is believed that this genetic response is activated through the introduction of the genetic cascade responsible for the osteoblast proliferation and subsequently promotes the increased rate of regeneration of hard tissue.

In certain further embodiments, the borate bioactive glass also contains silica as well as other ions such as sodium and calcium.

The preferred embodiment includes PGS as a carrier for melt and sol-gel derived borate bioactive glass. An exemplary borate bioactive glass is 45S5B1, in which the SiO₂ of 45S5 bioactive glass is replaced by B2O₃.

The 45S5B1 bioactive glass may be in a form of particles, spheres, fibers, mesh, sheets or a combination of these forms i.e. fibers within a sphere. The composition, porosity and particle sizes of the bioactive glass may vary.

The 45S5B1 bioactive glass may vary in size. For example, the particles of the 45S5B1 bioactive glass may range in size from 0.01 μm to 5 mm. Other ranges include about 1-5 micrometers, about 5-15 micrometers, about 15-50 micrometers, about 50-200 micrometers, about 200-1,000 micrometers, about 1-2 millimeters, about 2-3 millimeters, about 3-4 millimeters, or about 4-5 millimeters. In some embodiments, the bioactive glass particle has a diameter of between about 0.01 micrometer and about 5,000 micrometers.

In certain embodiments, the borate bioactive glass comprises 0-80%<100 μm, 0-80%<500 μm, 0-80% 500-1000 μm, 0-80% 1000-2000 μm, 0-80% 2000-5000 μm, 0-90% 90-710 μm, and 0-90% 32-125 μm bioactive glass.

The compositions may vary in molecular weight and may be blended in ratios of 10:1 up to 1:10.

Compositions of the glass may comprise from 0-90% boric acid with a plurality of other elements including Li, Na, K, Mg, Sr, Ti, Zr, Fe, Co, Cu, Zn, Al, Ga, P, N, S, F, Cl, and I. The embodiments take the consistency of a gel, putty, or waxy solid at room temperature.

Specifically, the borate bioactive glass material may have silica, sodium, calcium, strontium, phosphorous, as well as combinations thereof. In some embodiments, sodium, boron, strontium, and calcium may each be present in the compositions in an amount of about 1% to about 99%, based on the weight of the bioactive glass. In further embodiments, sodium, boron, strontium and calcium may each be present in the composition in at least about 1%, at least about 2%, at least about 3%, at least about 4%, at least about 5%, at least about 6%, at least about 7%, at least about 8%, at least about 9%, or at least about 10%.

In certain embodiments, silica, sodium, boron, and calcium may each be present in the composition in at least about 5 to about 10%, at least about 10 to about 15%, at least about 15 to about 20%, at least about 20 to about 25%, at least about 25 to about 30%, at least about 30 to about 35%, at least about 35 to about 40%, at least about 40 to about 45%, at least about 45 to about 50%, at least about 50 to about 55%, at least about 55 to about 60%, at least about 60 to about 65%, at least about 65 to about 70%, at least about 70 to about 75%, at least about 75 to about 80%, at least about 80 to about 85%, at least about 85 to about 90%, at least about 90 to about 95%, or at least about 95 to about 99%.

Some embodiments may contain substantially one or two of sodium, calcium, and strontium with only traces of the other(s). The term “about” as it relates to the amount of calcium phosphate present in the composition means +/−0.5%. Thus, about 5% means 5+/−0.5%.

The bioactive glass materials may further comprise one or more of a borosilicate, strontium, or calcium, including: SrO, CaO, P₂O₅. In certain embodiments, bioactive glass includes about 0-75% B₂O₃, 0-45% CaO, about 0-25% Na₂O, about 0-17% P₂O₅, about 0-30% MgO and about 0-5% CaF₂. One exemplary borate bioactive glass is 45S5B1, in which the SiO₂ of 45S5 bioactive glass is replaced by B₂O₃. 45S5B1 includes 46.1 mol % B₂O₃, 26.9 mol % CaO, 24.4 mol % Na₂O and 2.5 mol % P₂O₅. Other exemplary bioactive glasses include 58S, which includes 60 mol % SiO₂, 36 mol % CaO and 4 mol % P₂O₅, and S70C30, which includes 70 mol % SiO₂ and 30 mol % CaO.

In any of these or other bioactive glass materials of the invention, SrO may be substituted for CaO.

The following composition provided in Table 1 below, having a weight % of each element in oxide form in the range indicated, will provide one of several bioactive glass compositions that may be used to form a bioactive glass material:

TABLE 1

| | |
|---|---|
| CaO | 4-35 |
| Na₂O | 0-35 |
| P₂O₅ | 2-15 |
| CaF₂ | 0-25 |
| B₂O₃ | 0-75 |
| K₂O | 0-8 |
| MgO | 0-30 |
| CaF | 0-35 |

In case of the bioactive glass being in the form of a three-dimensional compressible body of loose glass-based fibers, the fibers may glass-formers such as P₂O₅, and B₂O₃. Some of the fibers have a diameter between about 100 nm and about 10,000 nm, and a length:width aspect ratio of at least about 10, The pH of the bioactive glass can be adjusted as-needed.

The bioactive glass particles, fibers, spheres, meshes or sheets may further comprise any one or more of adhesives, drafted bone tissue, in vitro-generated bone tissue, collagen, calcium phosphate, stabilizers, antibiotics, antibacterial agents, antimicrobials, drugs, pigments. X-ray contrast media, fillers, and other materials that facilitate grafting of bioactive glass to bone.

The silica and/or calcium ions released by the bioactive glass may improve the expression of osteostimulative genes. The borate and/or calcium ions may also increase the amount of and efficacy of proteins associated with such osteostimulative genes. In several embodiments, the bone repair material is osteostimulative and can bring about critical ion concentrations for the repair and regeneration of hard tissue without the necessity of any therapeutic materials or agents.

In some embodiments, the bone repair material is a composition comprising suspended autograft bone particles and suspended bioactive glass particles. Similar bone repair materials are described in U.S. Pub. No. 2015/0079146, the entire content of which is incorporated by reference.

In certain embodiments, the irrigation resistant bone repair material is a composition comprising suspended bioactive glass particles comprising B₂O₃. The composition may further include P₂O₅. PO₃, or PO₄. In some embodiments, the suspended bioactive glass particle may comprise 20-60% B₂O₃, 10-30% CaO, 0-4% P₂O₅, and 19-30% NaO. The suspended bioactive glass particle may further comprise a carrier selected from the group consisting of hydroxyapatite and tricalcium phosphate.

The borate bioactive glass particles, fibers, meshes or sheets may be pretreated in a solution comprising one or more of blood, bone marrow aspirate, bone-morphogenetic proteins, platelet-rich plasma, and osteogenic proteins. The proteins may be selected from the group consisting of WP9QY(W9; SEQ ID NO:1), OP3-4, RANKL, B2A, P1, P2, P3, P4, P24, P15, TP508, OGP, PTH, NBD, CCGRP, (Asp)₆ (SEQ ID NO:2), (Asp)₈ (SEQ ID NO:3), and (Asp, Ser, Ser)₆ (SEQ ID NO:4), and mixtures thereof.

In some embodiments, the bone repair material may be borate bioactive glass coated with a glycosaminoglycan, in which the glycosaminoglycan is bound to the bioactive glass. Bone repair materials incorporating glycosaminoglycans are described in U.S. Patent Pub. No. US 2014/0079789, the entire content of which is incorporated by reference. The glycosaminoglycan may be bound to the bioactive glass by means of an ionic bond or a covalent bond. The glycosaminoglycan may be heparin, heparan sulfate, chondroitin sulfate, dermatan sulfate, keratan sulfate, or hyaluronic acid.

In certain other embodiments, the bone repair material, such as borate bioactive glass may include surface immobilized peptides, as previously described in U.S. patent application Ser. No. 14/504,956, filed on Oct. 2, 2014, which is incorporated herein in its entirety.

In some further embodiments, the bone repair material is a bimodal bioactive glass composition comprising large borate bioactive glass particles and small borate bioactive glass particles. The large borate bioactive glass particles may have a substantially spherical shape and a mean diameter of between about 90 micrometers and about 2,000 micrometers. The small borate bioactive glass particles may have a substantially spherical shape and a mean diameter of between about 10 micrometers and about 500 micrometers.

In some embodiments, the borate bone repair material may be a trimodal bioactive glass composition comprising large borate bioactive glass particles, medium borate bioactive glass particles, and small borate bioactive glass particles. The large bioactive glass particles have a substantially spherical shape and a mean diameter of between about 500 micrometers and about 5,000 micrometers. The medium bioactive glass particles have a substantially spherical shape and a mean diameter of between about 90 micrometers and about 710 micrometers. The small bioactive glass particles have a substantially spherical shape and a mean diameter of between about 1 micrometers and about 125 micrometers.

In any of the above embodiments, small bioactive glass fibers may be added to the bone repair material. In certain embodiment, the small bioactive glass fibers may be borate bioactive glass fibers. The small bioactive glass fibers may have a diameter of less than 2 millimeters. The small bioactive glass fibers may be present in up to 40% by weight relative to the total weight of the bioactive glass. In various embodiments, the weight ratio of small bioactive glass fibers to total weight of the bioactive glass may be from 0-10%, 0-5%, 5-10%, 5-15%, 10-15%, 10-20%, 15-20%, 15-25%, 20-25%, 20-30%, 25-30%, 25-35%, 30-35%, 30-40%, or 35-40%. The weight ratio of small bioactive glass fibers to total weight of the bioactive glass may be about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%.

In some embodiments, any subset of the bioactive glass present, such as bioactive glass particles and/or small bioactive glass fibers, may be coated with silane as described in Verne et al. (Verne et al., “Surface functionalization of bioactive glasses,” J. Biomed. Mater. Res. A., 90(4):981-92 (2009)). The silane or other functional coatings may then allow for binding of proteins onto the bioactive glass, such as BMP-2.

In some embodiments, any subset of the bioactive glass present, such as bioactive glass particles and/or small bioactive glass fibers, may have additional silicate chains present on them. The additional silicate chains may allow the bioactive glass particles and fibers to interact with one another, as well as with groups of the non-ionic surfactants. The effect of these interactions may be to reduce the surface area of the filler, increase resin demand, and to allow for higher filler loadings.

In some embodiments, any subset of the bioactive glass present, such as bioactive glass particles and/or small bioactive glass fibers, may have added hydroxyl triethoxysilanes coated onto the glass. Some of these silanes are available from Gelest, Inc. For example, the glass may be coated with hydroxyl(polyethyleneoxy) propyltriethoxysilane. Additionally, the glass may be coated with other organic substituted ethoxy- and methoxy-silanes that are effective to create an interaction between the coated glass and the EO/PO carrier.

Certain further embodiments relate to an irrigation resistant bone repair composition that further includes at least one element selected from the group consisting of Li, Na, K, Mg, Sr, Ti, Zr, Fe, Co, Cu, Zn, Al, Ag, Au, Ce, Ga, P, N, S, F, Cl, Si and I. For example, small amounts of iodine, fluorine or silver can provide antimicrobial properties, while small amount of copper can promote angiogenesis (i.e., aid in the formation of blood vessels) while gold and cerium can reduce inflammation.

The irrigation resistant composition may further comprise ions and other compounds that may be dissolved in water. For example, the addition of salts, such as PBS, can enhance solidification and setting properties of PGS composition. Divalent salts may be particularly useful to improve the rheological properties of compositions containing PGS and bioactive glass materials as well as those of compositions containing PGS and other solid bone repair materials.

In certain embodiments, the composition may further include at least one additive, including but not limited to solvents, linear, straight chain and branched aliphatic hydrocarbons, sugars, polysaccharides, and hydroxyl or alkoxy terminal polyalkylene oxides along with low molecular weight biodegradable polymers (MW<=10,000).

Specific examples of additives include sodium hyaluronate, regenerez, polypropylene glycol 3000 (poly 3000), seasame oil, candelilla wax, carnauba wax, sorbitol (D-Glucitol), polycaprolactone, polycaprolactone diol, coconut oil, propylene glycol, polycaprolactone triol, polycaprolactone 10000 mw, mineral oil high viscosity, mineral oil low viscosity, polyethylene glycol 400 (PEG-8), butylene glycol, and hexylene glycol.

The biocompatible or bioactive bone repair material may be osteoinductive, osteoconductive, or a material that is both osteoinductive and osteoconductive. The bone repair material may be xenogeneic, allogeneic, autogeneic, and/or allo-plastic.

In certain embodiments, the irrigation resistant composition may further include a non-ionic surfactant, with the proviso that the non-ionic surfactant is not a non-random poly(oxyalkylene) block copolymer. The non-ionic surfactant or similar material other than the non-random poly(oxyalkylene) block copolymer may be selected from the group consisting of fatty acids (e.g. stearic acid), fatty alcohols (e.g., stearyl alcohol), alkoxylated alcohols (e.g., Ecosurf LF 45), alkoxylated alkylphenols (e.g., Triton X-100), alkoxylated fatty amides (e.g., polyethoxylated tallow amine), alkoxylated fatty esters (e.g., PEG 400 monostearate), alkoxylated fatty ethers (e.g., polyethylene glycol lauryl ether (Brij L23), polyglycerin fatty acid esters, alkoxylated sorbitan esters (e.g., Span 85 (sorbitan trioleate)), alkoxylated sorbitan esters (e.g., Polysorbate 20 and Polysorbate 80 also referred to as Tween 20 and Tween 80), fatty acid esters or polyol esters (e.g., glycerol monostearate, PEG coconut triglycerides), polyalkylene glycols (e.g., PEG 400 and PEG 600), alkoxylated organic acids, hydroxyacids or diacids and copolymers therefrom. Specific examples of non-ionic surfactants, other than the non-random poly(oxyalkylene) block copolymers, include sorbitan tristearate, polysorbate 20, polysorbate 80, polyoxyethylene 7 coconut, glycerides, poly(ethylene glycol) 400 monostearate (PEG 400 monostearate), PEG 2000 monomethylether, and PEG 400 distearate. Further examples of the non-ionic surfactants suitable for use with the irrigation resistant compositions include polyglyceryl-2 isostearate, polyglyceryl-2 diisostearate, polyglyceryl-4 isostearate, polyglyceryl-6 isostearate, poly(ethylene glycol) 8 stearate (MYRJ S8), polyglyceryl-10 isostearate, polyglyceryl-10 diisostearate, poly(ethylene glycol) 25 propylene glycol stearate (MYRJ S25), poly(ethylene glycol) 400 distearate (PEG 400 distearate), polyglyceryl-4 laurate, polyglyceryl-6 laurate, polyglyceryl-10 laurate, polyglyceryl-10 myristate, polyglyceryl-2 oleate, polyglyceryl-4 oleate, polyglyceryl-6 oleate, polyglyceryl-10 oleate, polyglyceryl-10 stearate, and polyglyceryl-10 distearate. Other non-ionic surfactants include polyoxyethylene 7 coconut glyceride (coconut glyceride), polyethylene glycol 2000 monomethyl ether (MME), glyceryl monostearate (monostearin), PEG dimethyl ether (dimethyl polyethylene glycol), PEG 200 adipate (poly(ethylene glycol) 200 adipate, PEG 6000 distearate, sorbitan monostearate, cetyl alcohol, ethylene glycol monostearate, propylene glycol stearate, polyoxyethylene stearyl ether (Brij 2), polyoxyethylene stearyl fatty ether (Brij 10), docosaethylene glycol mono octadecyl ether (Brij 20), polyethylene stearyl ether (Brij 100), polyglycerin fatty acid ester (polyglyceryl-2 isostearate, polyglyceryl-2 diisostearate, polyglyceryl-4 isostearate, polyglyceryl-6 isostearate, polyglyceryl-10 isostearate, polyglyceryl-10 diisostearate, polyglyceryl-4 laurate, polyglyceryl-6 laurate, polyglyceryl-10 laurate, polyglyceryl-10 myristate, polyglyceryl-2 oleate, polyglyceryl-4 oleate, polyglyceryl-6 oleate, polyglyceryl-10 oleate, polyglyceryl-10 stearate, polyglyceryl-10 distearate). Including the non-ionic surfactants into the irrigation resistant compositions allows to manipulate the melting point or cloud point above room temperature, and more preferably above a melting point above body temperature.

In any of the above embodiments, the irrigation resistant bone repair composition may be applied by a syringe at ambient temperature. After application to the bone or other site within the body at 37° C., the bone repair composition may harden or increase in viscosity and have a substantially lower tendency to migrate away from the application site.

More viscous bone repair compositions may be applied by painting the composition onto a site at or near the bone defect. Alternatively, more viscous bone repair compositions may be extruded onto the site in the form of a bead.

Methods

Certain embodiments relate to a method for treating hard tissues, such as bones using the irrigation resistant bone repair composition that includes (i) a porous biocompatible or bioactive bone repair material comprising borate, and (ii) poly(glycerol sebacate), wherein the composition, upon implantation into a surgical site, maintains position and does not displace upon irrigation of the surgical site.

Certain other embodiments relate to a method for treating a bone having a bone defect comprising contacting the bone at or near the site of the bone defect with the irrigation resistant bone repair composition of any of the above-described embodiments.

Any of the above-described materials or methods may be undertaken to treat any number of bone defects. As such, certain further embodiments relate to a method for treating a bone having a bone defect comprising placing an irrigation resistant bone repair composition of any one of the above-described embodiments at a site of a bone gap or a bone defect.

A bone defect may include bony structural disruptions, in which repair is needed or may be a gap in the bone or may arise from lack of adequate bone regeneration. A bone defect may be a void, which is understood to be a three-dimension defect that includes a gap, cavity, hole or other substantial disruption of the structural integrity of the bone or joint. The bone defects may also be fractures. The bone defects may also arise in the context of oral bone defects. The different types of bone defects are apparent to those of ordinary skill in the art. Gaps may be at least 2.5 cm and are generally in the range of 3-4 cm. This size is large enough so that spontaneous repair is not likely to occur and/or be complete. Exemplary bone defects include tumor resection, fresh fractures, cranial and facial abnormalities, spinal fusions, and loss of bone from the pelvis.

The various embodiments of the invention may be particularly useful with respect to orthopedic and spine processes because the material will stabilize and hold a better structure as it becomes more solidified when it heats up to body temperature.

Certain further embodiments relate to a method for treating a bone having a bone defect comprising placing an irrigation resistant bone repair composition of any one of the above-described embodiments at a bone gap or a bone defect.

In some embodiments, any of the above-described materials or methods may be combined with autograft bone chips for placement onto or near a bone defect. The materials may be a liquid or a gel at room temperature with the autograft bone chips suspended therein. Upon placement at or near the bone defect, the material will solidify around the autograft bone chips and serve to prevent the autograft bone chips from migrating away from the surgical sites.

In some embodiments, any of the above-described materials or methods may be combined with particles containing allogeneic or xenogeneic bone mineral for placement onto or near a bone defect. The materials may be a liquid or a gel at room temperature with the particles suspended therein. Upon placement at a surgical site, which is at or near the bone defect, the material will solidify around the particles and serve to prevent the particles from migrating away from the surgical site.

In various embodiments of the invention, the bone repair material is not a natural bone material or a synthetic bone material.

Kits

Further embodiments relate to kits that include an irrigation resistant bone repair composition including (i) a porous biocompatible or bioactive bone repair material comprising borate, and (ii) poly(glycerol sebacate), wherein the composition, upon implantation into a surgical site, maintains position and does not displace upon irrigation of the surgical site.

Exemplary kits for use with bone resistant compositions were previously described in U.S. Pub. No. US 2015/030684, which is incorporated herein in its entirety.

The kits may further include a dispensing gun, syringe, clam shell, adapter, plunger, tube(s), caps, assorted dispensing tips or other suitable delivery device and accompanying accessories. The irrigation resistant bone repair composition may be deposited into the tube(s) as part of the kit. An exemplary kit for delivery of other materials, such as Bioactive Synthetic Bone Graft Putty is currently being sold by NOVABONE® (NOVABONE® Bioactive Synthetic Bone Graft Putty MIS Cartridge Delivery System, NovaBone Products, LLC, Alachua, Fla.).

Exemplary Formulations:

Exemplary formulations are provided below. Generally, two primary types of formulations are preferred, PGS with borate glass and PGS with other carrier components, such as surfactants, cosolvents etc., and borate glass.

Formulations 1-3:

| | | | | BG | BG | BG | Poly |
|---|---|---|---|---|---|---|---|
| Formu- | | | Total | 1-2 | 90 μm- | 32 μm- | (glycerol |
| lation | Description | % BG | mass | mm | 710 μm | 125 μm | sebacate) |
| 1 | Poly(glycerol sebacate) only | 0 | 100.0 | 0.0 | 0.0 | 0.0 | 100.0 |
| 2 | All glass types | 75 | 100.0 | 50.0 | 12.5 | 12.5 | 25.0 |
| 3 | Without 1-2 mm glass | 71 | 100.0 | 0.0 | 57.5 | 13.5 | 29.0 |

Formulation 4:

| | | | | BG | BG | BG | | Sodium | Poly |
|---|---|---|---|---|---|---|---|---|---|
| Formu- | | | Total | 1-2 | 90 μm- | 32 μm- | MYRJ | Hyaluro- | (glycerol |
| lation | Description | % BG | mass | mm | 710 μm | 125 μm | S25 | nate | sebacate) |
| 4 | MYRJ s25 | 67 | 100.0 | 30.2 | 26.8 | 10.1 | 13.2 | 0.3 | 19.8 |

Formulations 5-7:

| | | | | BG | BG | BG | Poly | PEG | | | | Sodium |
|---|---|---|---|---|---|---|---|---|---|---|---|---|
| | | | Total | 1-2 | 90 μm- | 32 μm- | (glycerol | 400 | | Candelilla | Carnauba | Hyaluro- |
| Form. | Description | % BG | mass | mm | 710 μm | 125 μm | sebacate) | DI | Sorbitol | Wax | Wax | nate |
| 5 | Sorbitol | 73 | 100.0 | 0.0 | 58.8 | 13.7 | 6.0 | 15.2 | 6.0 | 0.0 | 0.0 | 0.3 |
| 6 | Candelilla wax | 73 | 100.0 | 0.0 | 58.8 | 13.7 | 6.0 | 15.2 | 0.0 | 6.0 | 0.0 | 0.3 |
| 7 | Carnauba wax | 73 | 100.0 | 0.0 | 58.8 | 13.7 | 6.0 | 15.2 | 0.0 | 0.0 | 6.0 | 0.3 |

Formulation 8:

| | | | BG | BG | BG | Poly | PEG | PEG | Sodium | |
|---|---|---|---|---|---|---|---|---|---|---|
| Formu- | | Total | 1-2 | 90 μm- | 32 μm- | (glycerol | 400 | 400 | Hyaluro- | Poly |
| lation | % BG | mass | mm | 710 μm | 125 μm | sebacate) | DI | MONO | nate | 3000* |
| PPG 3000 | 73 | 100 | 0.0 | 58.6 | 13.9 | 6.0 | 15.2 | 6.0 | 0.3 | 2.0 |

Formulations 9-12:

| | | | | BG | BG | Poly | PEG | | | | Sodium |
|---|---|---|---|---|---|---|---|---|---|---|---|
| Formu- | | | Total | 90 μm- | 32 μm- | (glycerol | 400 | Poly | Candelilla | | Hyaluro- |
| lation | Description | % BG | mass | 710 μm | 125 μm | sebacate) | DI | 3000* | Wax | Sorbitol | nate |
| 9 | PPG, Candelilla | 73 | 100 | 59.7 | 13.8 | 14.7 | 0.0 | 5.8 | 5.8 | 0.0 | 0.3 |
| 10 | PEG, PPG, | 73 | 100 | 59.7 | 13.8 | 7.8 | 7.8 | 6.8 | 3.7 | 0.0 | 0.3 |
| | Candelilla | | | | | | | | | | |
| 11 | PPG, Sorbitol | 73 | 100 | 59.7 | 13.8 | 14.7 | 0.0 | 5.8 | 0.0 | 5.8 | 0.3 |
| 12 | PEG, PPG, | 73 | 100 | 59.7 | 13.8 | 7.8 | 7.8 | 6.8 | 0.0 | 3.7 | 0.3 |
| | Sorbitol | | | | | | | | | | |

Formulations 13-16:

| | | | | BG | BG | PEG | Poly | | | | Sodium |
|---|---|---|---|---|---|---|---|---|---|---|---|
| Formu- | | | Total | 90 μm- | 32 μm- | 400 | (glycerol | Candelilla | Sesame | PCL | Hyaluro- |
| lation | Description | % BG | mass | 710 μm | 125 μm | DI | sebacate) | Wax | Oil | diol | nate |
| 13 | PEG, Sesame | 73 | 100 | 58.7 | 13.9 | 15.1 | 6.0 | 0.0 | 6.0 | 0.0 | 0.3 |
| 14 | PEG, Sesame | 73 | 100 | 57.5 | 13.6 | 14.9 | 11.7 | 0.0 | 1.9 | 0.0 | 0.3 |
| 15 | Candelilla | 73 | 100 | 59.1 | 14.0 | 0.0 | 14.5 | 6.0 | 6.0 | 0.0 | 0.3 |
| 16 | Sesame | 73 | 100 | 59.1 | 14.0 | 0.0 | 14.5 | 0.0 | 6.0 | 6.0 | 0.3 |

Formulations 17-18:

| | | | Total | Bioglass | Bioglass | Poly | Polycaprolactone | | | | Sodium |
|---|---|---|---|---|---|---|---|---|---|---|---|
| Formu- | | | sample | 90 μm- | 32 μm- | (glycerol | 10k | | | Sesame | Hyaluro- |
| lation | Description | % BG | mass | 710 μm | 125 μm | sebacate) | mw | Diol | Triol | Oil | nate |
| 17 | Polycaprolactone diol, | 73 | 100 | 59.1 | 14.0 | 14.5 | 0.0 | 6.0 | 6.0 | 0.0 | 0.3 |
| | triol | | | | | | | | | | |
| 18 | Polycaprolactone 10k | 73 | 100 | 59.1 | 14.0 | 14.5 | 6.0 | 0.0 | 6.0 | 0.0 | 0.3 |

Formulations 19-22:

| | | | | BG | BG | PEG | PEG | Poly | | | | Sodium |
|---|---|---|---|---|---|---|---|---|---|---|---|---|
| | | | Total | 90 μm- | 32 μm- | 400 | 400 | (glycerol | Candelilla | polycaprolactone | | Hyaluro- |
| Form. | Description | % BG | mass | 710 μm | 125 μm | MONO | DI | sebacate) | Wax | triol | diol | nate |
| 19 | PEG Di, Poly- | 73 | 100 | 58.7 | 13.9 | 0.0 | 15.1 | 6.0 | 0.0 | 6.0 | 0.0 | 0.3 |
| | caprolactone triol | | | | | | | | | | | |
| 20 | PEG Di, PEG | 73 | 100 | 57.5 | 13.6 | 5.8 | 14.9 | 5.8 | 0.0 | 1.9 | 0.0 | 0.3 |
| | Mono, Poly- | | | | | | | | | | | |
| | caprolactone triol | | | | | | | | | | | |
| 21 | PEG Mono, | 73 | 100 | 59.1 | 14.0 | 14.5 | 0.0 | 0.0 | 6.0 | 6.0 | 0.0 | 0.3 |
| | Candelilla, Poly- | | | | | | | | | | | |
| | caprolactone triol | | | | | | | | | | | |
| 22 | PEG Mono, Poly- | 73 | 100 | 59.1 | 14.0 | 14.5 | 0.0 | 0.0 | 0.0 | 6.0 | 6.0 | 0.3 |
| | caprolactone triol | | | | | | | | | | | |

Formulations 23-24:

| | | | | BG | BG | PEG | Poly | Mineral | Mineral | Sodium |
|---|---|---|---|---|---|---|---|---|---|---|
| Formu- | | | Total | 90 μm- | 32 μm- | 400 | (glycerol | Oil High | Oil Low | Hyaluro- |
| lation | Description | % BG | mass | 710 μm | 125 μm | DI | sebacate) | Viscosity | Viscosity | nate |
| 23 | Mineral Oil | 73 | 100 | 58.7 | 13.9 | 15.1 | 6.0 | 6.0 | 0.0 | 0.3 |
| | High Viscosity | | | | | | | | | |
| 24 | Mineral Oil | 73 | 100 | 58.7 | 13.9 | 15.1 | 6.0 | 0.0 | 6.0 | 0.3 |
| | Low Viscosity | | | | | | | | | |

Formulations 25-26:

| | | | | BG | BG | PEG | PEG | Poly | Mineral | Mineral | Sodium |
|---|---|---|---|---|---|---|---|---|---|---|---|
| Formu- | | | Total | 90 μm- | 32 μm- | 400 | 400 | (glycerol | Oil High | Oil Low | Hyaluro- |
| lation | Description | % BG | mass | 710 μm | 125 μm | MONO | DI | sebacate) | Viscosity | Viscosity | nate |
| 25 | PEG, Mineral Oil | 73 | 100 | 57.5 | 13.6 | 5.8 | 14.9 | 5.8 | 1.9 | 0.0 | 0.3 |
| | High Viscosity | | | | | | | | | | |
| 26 | PEG, Mineral Oil | 73 | 100 | 57.5 | 13.6 | 5.8 | 14.9 | 5.8 | 0.0 | 1.9 | 0.3 |
| | Low Viscosity | | | | | | | | | | |

Formulations 27-28:

| | | | | BG | BG | Poly | | Mineral | Mineral | Sodium |
|---|---|---|---|---|---|---|---|---|---|---|
| Formu- | | | Total | 90 μm- | 32 μm- | (glycerol | Candelilla | Oil High | Oil Low | Hyaluro- |
| lation | Description | % BG | mass | 710 μm | 125 μm | sebacate) | Wax | Viscosity | Viscosity | nate |
| 27 | Candelilla, Mineral | 73 | 100 | 59.1 | 14.0 | 14.5 | 6.0 | 6.0 | 0.0 | 0.3 |
| | Oil High Viscosity | | | | | | | | | |
| 28 | Candelilla, Mineral | 73 | 100 | 59.1 | 14.0 | 14.5 | 6.0 | 0.0 | 6.0 | 0.3 |
| | Oil Low Viscosity | | | | | | | | | |

Formulations 29-30:

| | | | | BG | BG | Poly | Mineral | Mineral | poly- | Sodium |
|---|---|---|---|---|---|---|---|---|---|---|
| Formu- | | | Total | 90 μm- | 32 μm- | (glycerol | Oil High | Oil Low | caprolactone | Hyaluro- |
| lation | Description | % BG | mass | 710 μm | 125 μm | sebacate) | Viscosity | Viscosity | diol | nate |
| 29 | Polycaprolactone diol, | 73 | 100 | 59.1 | 14.0 | 14.5 | 6.0 | 0.0 | 6.0 | 0.3 |
| | Mineral Oil High Viscosity | | | | | | | | | |
| 30 | Polycaprolactone diol, | 73 | 100 | 59.1 | 14.0 | 14.5 | 0.0 | 6.0 | 6.0 | 0.3 |
| | Mineral Oil Low Viscosity | | | | | | | | | |

Formulations 31-32:

| | | | | BG | BG | Poly | | | poly- | Sodium |
|---|---|---|---|---|---|---|---|---|---|---|
| Formu- | | | Total | 90 μm- | 32 μm- | (glycerol | Candelilla | Propylene | caprolactone | Hyaluro- |
| lation | Description | % BG | mass | 710 μm | 125 μm | sebacate) | Wax | Glycol | diol | nate |
| 31 | Candelilla, Propylene | 73 | 100 | 59.1 | 14.0 | 14.5 | 6.0 | 6.0 | 0.0 | 0.3 |
| | Glycol | | | | | | | | | |
| 32 | Propylene Glycol, Poly- | 73 | 100 | 59.1 | 14.0 | 14.5 | 0.0 | 6.0 | 6.0 | 0.3 |
| | caprolactone diol | | | | | | | | | |

Formulations 33-36:

| | | | | BG | BG | PEG | PEG | Poly | Mineral | | | Sodium |
|---|---|---|---|---|---|---|---|---|---|---|---|---|
| Formu- | | | Total | 90 μm- | 32 μm- | 400 | 400 | (glycerol | Oil High | Propylene | PCL | Hyaluro- |
| lation | Description | % BG | mass | 710 μm | 125 μm | MONO | DI | sebacate) | Viscosity | Glycol | diol | nate |
| 33 | PEG Mono, | 73 | 100 | 58.7 | 13.9 | 15.1 | 0.0 | 6.0 | 6.0 | 0.0 | 0.0 | 0.3 |
| | High Viscosity | | | | | | | | | | | |
| 34 | PEG Mono, PEG Di, | 73 | 100 | 58.7 | 13.9 | 11.1 | 4.0 | 6.0 | 6.0 | 0.0 | 0.0 | 0.3 |
| | High Viscosity | | | | | | | | | | | |
| 35 | PEG Di, Propylene | 73 | 100 | 59.1 | 14.0 | 0.0 | 14.5 | 0.0 | 0.0 | 6.0 | 6.0 | 0.3 |
| | Glycol | | | | | | | | | | | |
| 36 | PEG Mono, PEG Di, | 73 | 100 | 59.1 | 14.0 | 4.0 | 10.5 | 0.0 | 0.0 | 6.0 | 6.0 | 0.3 |
| | Propylene Glycol | | | | | | | | | | | |

Formulations 37-38:

| | | | | BG | BG | BG | BG | PEG | PEG | Poly | Sodium |
|---|---|---|---|---|---|---|---|---|---|---|---|
| | | | Total | 2-5 | 1-2 | 90 μm- | 32 μm- | 400 | 400 | (glycerol | Hyaluro- |
| Form. | Description | BG % | Mass | mm | mm | 710 μm | 125 μm | MONO | DI | sebacate) | nate |
| 37 | 2-5 mm, PEG | 63 | 100 | 47.6 | | | 15.5 | 8.1 | 20.7 | 8.1 | 0.3 |
| 38 | PEG | 67 | 100 | | 30.2 | 26.8 | 10.1 | 7.3 | 18.5 | 7.3 | 0.3 |

Formulation 16:

| | | | | BG | BG | PEG | PEG | Poly | | | | Sodium |
|---|---|---|---|---|---|---|---|---|---|---|---|---|
| | | | Total | 90 μm- | 32 μm- | 400 | 400 | (glycerol | Candelilla | Coconut | PCL | Hyaluro- |
| Form. | Description | % BG | Mass | 710 μm | 125 μm | MONO | DI | sebacate) | Wax | Oil | diol | nate |
| 39 | PEG Di, Coconut Oil | 73 | 100 | 58.7 | 13.9 | 0.0 | 15.1 | 6.0 | 0.0 | 6.0 | 0.0 | 0.3 |
| 40 | PEG Mono, PEG Di, | 73 | 100 | 57.5 | 13.6 | 5.8 | 14.9 | 5.8 | 0.0 | 1.9 | 0.0 | 0.3 |
| | Coconut Oil | | | | | | | | | | | |
| 41 | PEG Mono, Candelilla, | 73 | 100 | 59.1 | 14.0 | 14.5 | 0.0 | 0.0 | 6.0 | 6.0 | 0.0 | 0.3 |
| | Coconut Oil | | | | | | | | | | | |
| 42 | PEG Mono, Coconut | 73 | 100 | 59.1 | 14.0 | 14.5 | 0.0 | 0.0 | 0.0 | 6.0 | 6.0 | 0.3 |
| | Oil, PCL diol | | | | | | | | | | | |

Throughout this specification various indications have been given as to preferred and alternative embodiments of the invention. However, the foregoing detailed description is to be regarded as illustrative rather than limiting and the invention is not limited to any one of the provided embodiments. It should be understood that it is the appended claims, including all equivalents, are intended to define the spirit and scope of this invention.

1. A bone repair composition comprising:
a porous biocompatible or bioactive bone repair material comprising
borate, and
poly(glycerol sebacate),
wherein the composition, upon implantation into a surgical site, maintains position and does not displace upon irrigation of the surgical site.

2. The bone repair composition of claim 1, wherein the weight ratio of the poly(glycerol sebacate) is 1%-99% relative to the weight of the bone repair composition.

3. The bone repair composition of claim 1, wherein the composition is osteoconductive.

4. The bone repair composition of claim 1, wherein the composition is osteostimulative.

5. The bone repair composition of claim 1, wherein the bone repair material is a borate bioactive glass or ceramic.

6. The bone repair composition of claim 5, wherein the borate bioactive glass is a melt-derived borate bioactive glass or a sol-gel derived borate bioactive glass.

7. The bone repair composition of claim 6, wherein the bioactive glass is in the form of a particle, sphere, fiber, mesh, sheet or a combination of these forms.

8. The bone repair composition of claim 6, wherein the bioactive glass is in the amorphous or crystalline form, or a combination of these forms.

9. The bone repair composition of claim 5, wherein the composition forms a synthetic bone-grafting putty, paste, gel, strip or waxy solid.

10. The bone repair composition of claim 5, wherein the composition is in an injectable form.

11. The bone repair composition of claim 1, wherein the composition promotes osteointegration upon introduction into a bony defect.

12. The bone repair composition of claim 6, wherein the bioactive glass comprises about 10-45% CaO, about 10-70% B₂O₃, about 0-25% Na₂O, about 0-17% P₂O₅, about 0-30% MgO and about 0-5% CaF₂.

13. The bone repair composition of claim 6, wherein the bioactive glass comprises about 45% B₂O₃, about 24.5% CaO, about 6% P₂O₅, and about 2.5% Na₂O.

14. The bone repair composition of claim 7, wherein the size of the bioactive glass particle is in a range from about 0.01 μm to about 5 mm.

15. The bone repair composition of claim 5, wherein the borate bioactive glass comprises 0-80%<100 μm bioactive glass, 0-80%<500 μm bioactive glass, 0-80% 500-1000 μm bioactive glass, 0-80% 1000-2000 μm bioactive glass, 0-80% 2000-5000 μm bioactive glass, 0-90% 90-710 μm bioactive glass, and 0-90% 32-125 μm bioactive glass.

16. The bone repair composition of claim 7, wherein the bone repair material is one or more particles of bioactive glass coated with a glycosaminoglycan, wherein the glycosaminoglycan is bound to the bioactive glass.

17. The bone repair composition of claim 1, further comprising at least one element selected from the group consisting of Li, K, Mg, Sr, Ti, Zr, Fe, Co, Cu, Zn, Al, Ag, Au, Ce, Ga, P, N, S, F, Cl, Si and I.

18. The bone repair composition of claim 1, wherein the composition comprises a bioactive agent.

19. The bone repair composition of claim 18, wherein the bioactive agent comprises a bioactive glass ceramic comprising silica and boron.

20. The bone composition of claim 1, wherein the composition is in a flexible-crosslinked form in a shape of a strip.

21. The bone repair composition of claim 1, further comprising an additive selected from the group consisting of a solvent, a linear aliphatic hydrocarbon, straight chain aliphatic hydrocarbon, branched aliphatic hydrocarbon, sugar, polysaccharide, and hydroxyl terminal polyalkylene oxide, alkoxy terminal polyalkylene oxide, and a low molecular weight biodegradable polymers (MW</=10,000).

22. The bone repair composition of claim 21, wherein the additive is selected from the group consisting of sodium hyaluronate, Regenerez® (polyglycerol sebacate), polypropylene glycol 3000 (poly 3000), seasame oil, candelilla wax, carnauba wax, sorbitol (D-Glucitol), polycaprolactone, polycaprolactone diol, coconut oil, propylene glycol, polycaprolactone triol, polycaprolactone 10000 mw, mineral oil high viscosity, mineral oil low viscosity, polyethylene glycol 400, butylene glycol, and hexylene glycol.

23. An irrigation resistant putty or paste including the composition of claim 1 mixed with water, saline, blood, or BMA.

24. The bone repair composition of claim 1, wherein the composition is for treating a bone defect or a bone gap.

25. The bone repair composition claim 1, wherein the composition is for regeneration of hard tissues.

26. A method for treating a bone having a bone gap or a bone defect comprising contacting the bone at or near the site of the bone defect with the bone repair composition of claim 1.


SEQUENCE LISTING (4 sequences)

SEQ ID NO: 1 - protein, 9 aa, Artificial Sequence
  artificial peptide, bone resorption inhibitor, WP9QY(W9)
YCWSQYLCY

SEQ ID NO: 2 - protein, 6 aa, Artificial Sequence
  artificial bone targeting peptide
DDDDDD

SEQ ID NO: 3 - protein, 8 aa, Artificial Sequence
  artificial bone targeting peptide
DDDDDDDD

SEQ ID NO: 4 - protein, 18 aa, Artificial Sequence
  artificial bone targeting peptide
DSSDSSDSSDSSDSSDSS